(19) United States
(12) Patent Application Publication
(10) Pub. No.: US 2010/0086442 A1
(43) Pub. Date: Apr. 8, 2010

(54) Biosensor to Detect Infectious Agents

(75) Inventors: Robert Kirken, El Paso, TX (US); Eric MacDonald, El Paso, TX (US); Ryan Wicker, El Paso, TX (US); Kristin Gosselink, El Paso, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/573,100

(22) Filed: Oct. 3, 2009

Related U.S. Application Data
(60) Provisional application No. 61/102,748, filed on Oct. 3, 2008.

(51) Int. Cl. G01N 33/53

(52) U.S. Cl. 422/69

(57) ABSTRACT

The present invention provides a sensor for the detection of chemical agents in real time. The sensor includes a sample region having a detector disposed about a substrate where a chemical agent binds to the detector to generate in a signal. The sensor includes a microcontroller in communication with the detector, an analog to digital converter, and a communication module to process the signal into an analytical signal. In addition, the sensor includes an antenna in communication with the microcontroller to enable a wireless communication network, wherein the analytical signal can be transmitted and a power source in operable communication with the microcontroller to supply a source of power to the sensor.

### CROSS-REFERENCE TO RELATED APPLICATIONS

This application claims priority to U.S. Provisional Application Ser. No. 61/102,748, filed Oct. 3, 2008, the entire contents of which are incorporated herein by reference.

### TECHNICAL FIELD OF THE INVENTION

The present invention relates in general to the field of detecting allergens, chemical agents, proteins and biological warfare agents.

### BACKGROUND OF THE INVENTION

Without limiting the scope of the invention, its background is described in connection with detection devices, systems and kits for the detection of various allergens and biological agents. Currently, rapid detection and identification of various biological agents, such as anthrax, is an ongoing issue for national security and health due to their capacity to infect and overwhelm humans. The method of transmission to these agents allows widespread dispersion of the agents in a limited amount of time. The capability to predict the spread in order to minimize the threat, isolate infected individuals and provide adequate medical response in a time-dependent manner is an ongoing concern.

For example, U.S. Pat. No. 7,430,046 discloses a particle detector with a sample area of cross section no in excess of about 2 mm for containing environmental fluid, a light source on one side of the sample area for directing a collimated or nearly collimated beam of light through the sample air or water so that part of the light beam will be scattered by any particles present in the air or water while the remainder remains unscattered, and a beam diverting device on the opposite side of the sample area for diverting or blocking at least the unscattered portion of the beam of light and directing at least part of the scattered light onto a detector. The detector produces output pulses in which each pulse has a height proportional to particle size and a pulse height discriminator obtains the size distribution of airborne particles detected in the air or water sample at a given time from the detector output. The detector may also include a device for discriminating between biological agents and inorganic particles.

### SUMMARY OF THE INVENTION

The present invention provides an apparatus and method for the detection of biological agents, such as anthrax, potentially transmitted though natural and unnatural delivery routes. The present invention allows monitoring pathogens. The present invention provides enhanced detection sensitivity and reduced analysis time.

The present invention provides a sensor for the detection of chemical agents in real time. The sensor includes a sample detection region disposed on or within a substrate comprising an antibody or receptor, wherein a chemical agent binds to the antibody or receptor in contact with a signal generator; a microcontroller in communication with the signal generator, the microcontroller further comprising an analog to digital converter and a communication module to process the signal into an analytical signal; an antenna in communication with the microcontroller to enable communication with a wireless communication network, wherein the signal obtained from the sample detection region is processed and transmitted; and a power source in operable communication with the microcontroller to supply a source of power to the sensor, wherein the sensor, the microcontroller, the antenna and the power source are embedded in the substrate.

The present invention also includes an apparatus for immunochemical detection of an analyte. The apparatus includes one or more antibodies immobilized in a substrate and configured to transmit a signal upon binding of a target, wherein the one or more antibodies comprises at least a single antibody variable heavy chain or a single-chain Fv polypeptide specific for Bacillus anthracis, a microcontroller to receive the signal, wherein the microcontroller comprises an analog to digital converter, a signal amplifier, a noise reduction unit, a storage device, a microprocessor or a combination thereof to process the signal, a power source in operable communication with the microcontroller to supply a source of power to the sensor, and a communication module in communication with the microcontroller to transmit the signal to a remote control center. In one embodiment, the one or more antibodies are connected to the microcontroller through a piezoelectric device. In one aspect, the antibody or receptor is connected to the signal generator via a MEMS or a piezoelectric device.

Yet another embodiment of the present invention includes an apparatus for immunochemical detection of Bacillus anthracis, comprising: monoclonal antibodies specific for Bacillus anthracis immobilized in a polymeric substrate and connected to a sensor by a piezoelectric device; a small efficient antenna disposed on the polymeric substrate and connected to the sensor for the transmission of information to a gateway device, cell tower or satellite, a GPS chip set disposed on the polymeric substrate and connected to the antenna to provide self-location information of the apparatus, a microcontroller connected to the sensor to receive a signal, wherein the microcontroller comprises an analog to digital converter, a signal amplifier, a noise reduction unit, a storage device, a microprocessor or a combination thereof to process the signal; a power source in operable communication with the microcontroller to supply a source of power to the sensor; and a communication module in communication with the microcontroller to transmit the signal to a remote control center. In one aspect, the power source comprises a battery, a solar power system, a direct power connection, an antenna to receive power from another source or a combination thereof. In another aspect, the antenna can send, receive or send and receive signals in the form of wifi, RFID, blue tooth, RF, IR, AM, FM, light, or a combination thereof. In another aspect, the communication module comprises a MEMS, piezoelectric device, a RFID code-able chip or a combination thereof.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic of one embodiment of the integrated sensor system for pathogen of the present invention;

FIG. 2 is an image of one embodiment of the SFF electrical structures of the present invention; and

FIG. 3 is a flowchart of the infectious biosensor detector of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “antibody” is used in the broadest sense unless clearly indicated otherwise. Therefore, an “antibody” can be naturally occurring or man-made such as monoclonal antibodies produced by conventional hybridoma technology and antibodies comprise monoclonal and polyclonal antibodies as well as fragments containing the antigen-binding domain and/or one or more complementarity determining regions of these antibodies. As used herein, the term “antibody” refers to any form of antibody or fragment thereof that specifically binds the target and/or exhibits the desired biological activity and specifically covers monoclonal antibodies (including full length monoclonal antibodies), polyclonal antibodies, multispecific antibodies (e.g., bispecific antibodies), and antibody fragments so long as they specifically bind and/or exhibit the desired biological activity. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that form a specific binding site for the target antigen. The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, apes.

As used herein, the term “receptor” refers to a specific binding partner of a ligand and includes, without limitation, membrane receptors, soluble receptors, cloned receptors, recombinant receptors, hormone receptors, drug receptors, transmitter receptors, autocoid receptors, cytokine receptors, antibodies, antibody fragments, engineered antibodies, antibody mimics, molecular recognition units, adhesion molecules, agglutinins, integrins, selectins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule. In one embodiment, the receptor may be a lectin that binds to specific surface polysaccharides of an infectious agent.

In this regard, the present invention provides embodiments for detecting infections agents includes, e.g., viruses, bacteria, fungi or mycoplasma. The present invention in not limited, however, to treating any particular infection or to the destruction of any particular infectious agent. For example, in some embodiments, the present invention provides compositions directed to treating (e.g., mediating the translocation of a therapeutic agents) to ameliorate diseases caused by the following exemplary pathogens: Bartonella henselae, Borrelia burgdorferi, Campylobacter jejuni, Campylobacter fetus, Chlamydia trachomatis, Chlamydia pneumoniae, Chylamydia psittaci, Simkania negevensis, Escherichia coli (e.g., 0 1 57:H7 and K88), Ehrlichia chafeensis, Clostridium botulinum, Clostridium perfringens, Clostridium tetani, Enterococcusfaecalis, Haemophilus influenzae, Haemophilus ducreyi, Coccidioides immitis, Bordetella pertussis, Coxiella burnetii, Ureaplasma urealyticum, Mycoplasma genitalium, Trichomatis vaginalis, Helicobacterpylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacteriumfortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracis, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptoccus, Cryptosporidum, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, Encephalitozoon cuniculi.

The present invention provides a sensitive biosensor that is independent of human operators and coupled with wireless communication capabilities. One embodiment of the present invention provides a biological sensor highly specific for Bacillus anthrax. A new, high sensitivity biological sensor includes substrate impregnated with high-affinity monoclonal antibodies or “anthrax receptors” capable of distinguishing the pathogen. The biological sensor further includes a polymer substrate that has been designed to deliver samples to the sensor, resist extreme environmental conditions and integrate the necessary electronics for sensing and wireless communication. The present invention may be fabricated using solid freeform fabrication technologies.

A novel and crucial defense and homeland security application of the combined technology of solid freeform fabrication, coupled with the dispensing of conductive inks, is a wireless sensor system. This technology will allow for the placement of simple monitoring stations within cities, along borders, or other areas deemed of “critical sensitivity”. In addition the present invention also includes a hand held devices are also possible.

The design freedom provided by solid freeform fabrication allows for the fabrication of substrates that are intricately-detailed with an arbitrary 3D shape and, as such, can be used to create micro fluidic channels within the structure providing directed air flow over the proposed sensor. Solid freeform fabrication is a collection of techniques for manufacturing solid objects by the sequential delivery of energy and/or material to specified points in space to produce that solid. For example, the solid freeform fabrication allows rapid prototyping using virtual designs from computer aided design (CAD) or modeling software, transforms them into thin, virtual, horizontal cross-sections and then creates each cross-section in physical space, one after the next until the model is finished. The machine reads in data from a CAD drawing and lays down successive layers of liquid, powder, or sheet material, and in this way builds up the model from a series of cross sections. These layers, which correspond to the virtual cross section from the CAD model, are joined together or fused automatically to create the final shape. The primary advantage to additive fabrication is its ability to create almost any shape or geometric feature. Other solid freeform fabrication techniques use two materials in the course of constructing parts. The first material is the part material and the second is the support material to support overhanging features during construction. The support material is later removed by heat or dissolved away with a solvent or water. Injection molding can also be used to construct the present invention.

The present invention provides a sensor for the detection of chemical agents in real time. The sensor includes a sample region having a detector disposed about a substrate where a chemical agent binds to the detector to generate in a signal. The sensor includes a microcontroller in communication with the detector, an analog to digital converter, and a communication module to process the signal into an analytical signal. In addition, the sensor includes an antenna in communication with the microcontroller to enable a wireless communication network, wherein the analytical signal can be transmitted and a power source in operable communication with the microcontroller to supply a source of power to the sensor.

The detector may include one or more high-affinity monoclonal antibodies. Although the skilled artisan will readily understand that other antibodies, proteins, polypeptides, nucleic acids, receptors, binding agents, or other compositions that specifically bind to a specific agent. Although one embodiment includes the chemical target as Bacillus anthracis (anthrax), other embodiments may be for chemicals, gases, particles, and so forth. The sensor may include a detector having a plurality of sensors, each said plurality of sensors configured to enable detection of a different chemical target. These sensors may be in distinct regions or dispersed throughout the substrate.

The power source may be in various forms and may be integrated in to the substrate in some embodiments and may include a battery, a solar power system, a direct power connection, an antenna or receiver to receive power from another source or a combination thereof. The sensor may include an antenna to send, receive or send and receive signals in the form of wifi, RFID, blue tooth, RF, IR, AM, FM, light, or a combination thereof and/or a GPS device. In some embodiments, the power supplied to the device may be supplied from an external source. The communication module may be a micro-electronic mechanical system (MEMS), piezoelectric device, a RFID code-able chip or a combination thereof and may transmit and/or receive analog signals, digital signals or both to a remote control center. The remote control center may be local or at a distant location or may be integrated into a wearable item, button, clothing, garment, belt, or other garment or integrated into a larger device.

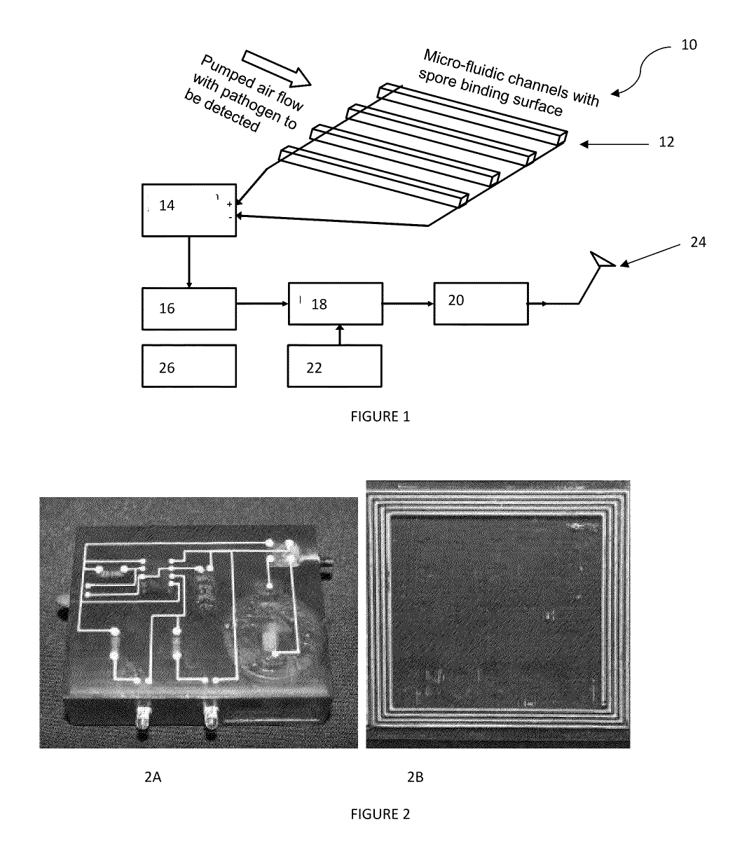

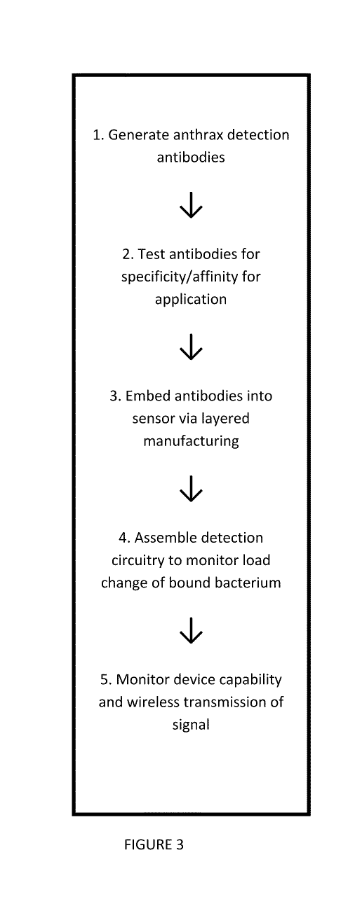

For example, FIG. 1 illustrates one embodiment of the present invention. The detector 10 includes a sample region 12. The sample region may have different shapes, areas, surface, textures, dividers, channels, ridges, pooling areas, buffers, blockers, diffusers, and so forth. The sample region 12 is in communication with a instrument 14 to receive the signals from the detection. This communication may be a hard connection through wiring or a wireless connection (wifi, RFID, blue tooth, RF, IR, etc.) or even a combination there of. The instrument 14 may include numerous components including an analog to digital converter 16, a microcontroller 18, a communication system 20 and an optional GPS 22. Depending on the communication system the instrument 14 may include an antenna 24. The communication system 20 may be designed to operate using wifi, RFID, blue tooth, RF, IR, etc. The instrument 14 also includes a power source 26. The power source 24 may be a RFID power source, a battery, a solar power system, a direct power connection, or may receive power from another source.

The present invention includes conductive traces drawn by advanced dispensing technology, the sensor area is electrically monitored and the generated signal is connected to the appropriate infrastructure of electronics required to collect and deliver the data in a timely fashion. This infrastructure of the present invention includes a small efficient antenna for the transmission of information to a gateway device, cell tower or satellite. The present invention includes a GPS chip set in order to provide self-location information of the device. The present invention also includes a microcontroller to filter the data and prevent false positives and a wireless communication sub-system to communicate details of the detection. Furthermore, other supporting electronics can be tightly integrated into the interior of the substrate to improve system size and power consumption.

FIG. 2 is an image of a solid freeform fabrication sensor. The capability of fabricating the system in 3D also opens up the possibility of implementing these devices not only as unattended ground sensors, but as light-weight and rugged components in manned or unmanned aerial vehicles as well, thus providing for a range multiplier for the detection of the pestilent bacterium.

In addition, the overall strength of the circuitry could be increased by embedding the electronic components and circuitry within solid structural members, as compared to traditional PCB designs, protecting the electronics from outside hazards. Components tightly integrated into an arbitrarily formed substrate will be naturally resistant to reverse engineering and the relatively low cost will allow for potential mass deployment.

The present invention provides a method and apparatus to identify highly selective and chemically pure biosensors that detect anthrax with high affinity by relying on monoclonal antibody technology. The present invention provides unique domains within anthrax as targets for generating highly sensitive antibody based detection that will likely target exosporium gene products, e.g., Bcl A, Bxp A, etc.). The present invention includes multiple and redundant sites to ensure positive readings and minimize false positives. The present invention detects exosporium gene products. The present invention may be used to detect bacterial spores, such as Bacillus anthracis (anthrax), Clostridium tetani (tetanus), and Clostridium botulinum (botulism). Additionally, the present invention includes mice and other mammals immunized with intact anthrax spores to generate these high affinity antibodies. In addition, the present invention may be used to detect chemical compounds, virus, bacteria, isotopes, nucleic acids, proteins, peptides, and combinations thereof.

For example, U.S. Pat. No. 7,329,536, incorporated herein by reference, discloses an apparatus comprising one or more piezoelectric mass sensors for use in diagnostic and analytic processes, in particular for immunochemical detection of diagnostically relevant analytes in real time, is described. Each piezoelectric mass sensor comprises a piezoelectric crystal with a receptor surface which has immobilized thereon a lawn of recombinant antibodies comprising single VH chain or single-chain Fv polypeptides specific for a particular antigen. Binding of antigen to the recombinant antibodies results in a change in mass on the receptor surface which is detected as a change in resonant frequency.

U.S. Pat. No. 7,271,720, incorporated herein by reference, discloses nano-sensors embedded in a silicon substrate and etched/fused in a micro-fibered material to enable an outfit for monitoring suspicious terrorist activities and for track biological and chemical gases, and explosives, including stationary and portable weapons of mass destruction. Detected signals are transported wirelessly through radio frequency signals to a central security monitoring station, enabling communication with first responders and backup security personnel or agents to the vicinity of the detection. The sensors are multifunctional and coded to recognize wavelike pattern of gases and explosives traveling through wave. The wired outfit and the receptor are operable to process the portion of the detection signal to determine whether there is a concealed object by conducting a test in which a first characteristic of a first dielectric constant associated with a person is determined, and a second characteristic of a second dielectric constant associated with the concealed object and or weapons of mass destruction is determined to expedite data transmission and communication to first responders.

The binding of the associated antibody/antigen caused by specific recognition would result in mass increase and decrease in frequency. The change of frequency reflects the presence and amount of the targets. Achievement is obtained through manipulation of the structure by controlling the deposition parameter precisely.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A sensor for the detection of chemical agents in real-time comprising:
a sample detection region disposed on or within a substrate comprising an antibody or receptor, wherein a chemical agent binds to the antibody or receptor in contact with a signal generator;
a microcontroller in communication with the signal generator, the microcontroller further comprising an analog to digital converter and a communication module to process the signal into an analytical signal;
an antenna in communication with the microcontroller to enable communication with a wireless communication network, wherein the signal obtained from the sample detection region is processed and transmitted; and
a power source in operable communication with the microcontroller to supply a source of power to the sensor, wherein the sensor, the microcontroller, the antenna and the power source are embedded in the substrate.

2. The sensor of claim 1, wherein the antibody comprises one or more high-affinity monoclonal antibodies.

3. The sensor of claim 1, wherein the chemical agent comprises Bacillus anthracis (anthrax).

4. The sensor of claim 1, wherein the power source comprises a battery, a solar power system, a direct power connection, an antenna to receive power from another source or a combination thereof.

5. The sensor of claim 1, wherein the antenna sends, receives or sends and receives signals in the form of wife, RFID, blue tooth, RF, IR, AM, FM, light, or a combination thereof.

6. The sensor of claim 1, further comprising a GPS device in communication with the microcontroller.

7. The sensor of claim 1, wherein the substrate comprises metal, polymer, composite or combinations thereof.

8. The sensor of claim 1, wherein the sample detection region comprises a plurality of sensors, each said plurality of sensors configured to enable detection of a different chemical agent.

9. The sensor of claim 1, wherein the antibody or receptor is connected to the signal generator via a micro-electronic mechanical system (MEMS) or a piezoelectric device.

10. The sensor of claim 1, further comprising a memory and data storage device in communication with the microcontroller.

11. The sensor of claim 1, wherein the communication module transmits and receives analog signals, digital signals or both.

12. The sensor of claim 1, wherein the communication module transmits the analytical signal to a remote control center.

13. The sensor of claim 1, further comprising an operational amplifier circuit in communication with the detector to amplify the signal.

14. The sensor of claim 1, wherein the sensor is integrated into a wearable item.

15. An apparatus for immunochemical detection of an analyte, comprising:
one or more antibodies immobilized in a substrate and configured to transmit a signal upon binding of a target, wherein the one or more antibodies comprises at least a single antibody variable heavy chain or a single-chain Fv polypeptide specific for Bacillus anthracis;
a microcontroller to receive the signal, wherein the microcontroller comprises an analog to digital converter, a signal amplifier, a noise reduction unit, a storage device, a microprocessor or a combination thereof to process the signal;
a power source in operable communication with the microcontroller to supply a source of power to the sensor;
a communication module in communication with the microcontroller to transmit the signal to a remote control center.

16. The apparatus of claim 15, wherein the one or more antibodies are connected to the microcontroller through a piezoelectric device.

17. An apparatus for immunochemical detection of Bacillus anthracis, comprising:
monoclonal antibodies specific for Bacillus anthracis immobilized in a polymeric substrate and connected to a sensor by a piezoelectric device or MEMS;
a small efficient antenna disposed on the polymeric substrate and connected to the sensor for the transmission of information to a gateway device, cell tower or satellite,
a GPS chip set disposed on the polymeric substrate and connected to the antenna to provide self-location information of the apparatus,
a microcontroller connected to the sensor to receive a signal, wherein the microcontroller comprises an analog to digital converter, a signal amplifier, a noise reduction unit, a storage device, a microprocessor or a combination thereof to process the signal;
a power source in operable communication with the microcontroller to supply a source of power to the sensor; and
a communication module in communication with the microcontroller to transmit the signal to a remote control center.

18. The apparatus of claim 17, wherein the power source comprises a battery, a solar power system, a direct power connection, an antenna to receive power from another source or a combination thereof.

19. The apparatus of claim 17, wherein the antenna can send, receive or send and receive signals in the form of wifi, RFID, blue tooth, RF, IR, AM, FM, light, or a combination thereof.

20. The apparatus of claim 17, wherein the communication module comprises a MEMS, piezoelectric device, a RFID code-able chip or a combination thereof.
